(19) United States
(12) Patent Application Publication
(10) Pub. No.: US 2017/0295843 A1
(43) Pub. Date: Oct. 19, 2017

(54) Advanced Herb Vaporizing Prevents Burning/Singeing & Facilitates French Inhaling: Thingy™ & CozyNosie™

(71) Applicant: Leonard Storch, New York, NY (US)

(72) Inventor: Leonard Storch, New York, NY (US)

(21) Appl. No.: 15/470,189

(22) Filed: Mar. 27, 2017

Related U.S. Application Data
(60) Provisional application No. 62/324,533, filed on Apr. 19, 2016.
(60) Provisional application No. 62/452,432, filed on Jan. 31, 2017.

(51) Int. Cl. A24F 47/00; A61M 15/00

(52) U.S. Cl. CPC A24F 47/008; A61M 15/0021

(57) ABSTRACT

Often when vaporizing herbs like marijuana, the pot gets singed or burned creating smoke; inhaling smoke is unhealthy. An improved vaporizer mode & apparatus allowing a non-burning gas, such as carbon dioxide or nitrogen from small readily-available cartridges, to pass over the heated pot to inhale the herb's vapor instead of air to avoid the risk of the herb being singed or burned which makes smoke amidst the chemical vapor. Without air's oxygen, burning and smoke are just not possible no matter how dry the pot is or how hot the pot gets.

Furthermore, finally, apparatus to facilitate taking a French inhale from an herb vaporizer has been invented! French inhaling is cool, the tobacco or other herb “tastes” and feels more enjoyable (most “flavor” is in the nose) and French inhaling prevents herbal-vapor from “fouling” the user's mouth.

Vapor carried by a non-burning gas may be French inhaled.

### CROSS-REFERENCE TO RELATED U.S. APPLICATION

This application is a relation of Provisional Application No. 62/452,432 filed Jan. 31, 2017 titled “Nosie, French Inhaling Vaporizing Accessory, e.g., w/a Pax2,” and this application is a relation of Provisional Application No. 62/324,533 filed Apr. 19, 2016 titled “Healthier & Happier Marijuana Vaporizing Thingys™,” and the '432 & '533 applications are incorporated herein by reference.

### FIELD OF ART

The field of art is the use of vaporizers and vaporizer accessories for herbs such as marijuana and tobacco. While vaporizers are also used for other herbs, the disclosure herein uses marijuana by way of example; the present invention is not meant to be limited to marijuana or tobacco.

Musings: Since the beginning, from societal and business views, this field of art has been and continues to be extremely controversial. Many have viewed in the past, and/or still view, tobacco and/or marijuana with abject hate while, ironically, many others are inclined to view tobacco and/or marijuana most favorably. The remarkable irony of such contrasting views remains controversial as this field of art evolves with the use of modern technology offering alternative delivery systems, and accessories therefor, for herbs.

### BACKGROUND

Complementary embodiments of the present vaporizer invention are disclosed. Given the health & happiness effects of marijuana, people have been smoking it for eons. The inventor herein gave up smoking marijuana when diagnosed with COPD to avoid further damage to his lungs because, as with tobacco smoke, pot smoke is harmful to lungs. Smoke is the result of burning or singeing herb, and oxygen is required for burning or singeing. This inventor ventures this personal observation: Remarkably, inhaling psychotropic components of marijuana vapor helps me breathe. While COPD is a physical lung condition, COPD also produces anxiety; marijuana vapor allays anxiety. (Why marijuana helps my breathing is related to autonomic nervous systems & why water-boarding works, but that is of no present moment.)

What is relevant at the moment is the fact that many vaporizer users, including myself, have noticed that pot may burn or singe (“singe” meaning to burn (something) superficially) or smolder (smolder, meaning to smoke but not flame) in different vaporizers that are available—the Internet has many articles about this subject, i.e., about pot burning in vaporizers producing smoke. You can tell sometimes how singed or burned the herb is by looking for tell-tale signs including a dark color or burnt color after vaporizing. Factors involved with singeing and burning are discussed below.

According to the “The law of conservation of mass”, a discovery attributed to Antoine-Laurent de Lavoisier, a French nobleman and chemist central to the 18th-century chemical revolution, combustion (burning) requires oxygen. Without oxygen, burning pot is not possible, and therefore smoke is not possible. Air is about 20% oxygen O₂ and about 80% nitrogen N₂. If a vaporizer could operate without air, burning (combustion) and smoke would be avoided. Combustion is involved in making smoke, but should not be involved in making vapor.

As disclosed, vaporizers may harmlessly use gases other than air, for example, gases such as nitrogen N₂ or carbon dioxide CO₂. Such inert gases do not support burning, are readily commercially available in convenient sized cartridges and are not harmful to inhale for vaporizing (perhaps comparable to holding one's breath for several seconds (after all, air is 80% N₂)).

In the recent few years, vaporizers have become very popular among marijuana users. Vaporizers come in all shapes, sizes and varieties, some for example are called pens and some are housed in wooden boxes, and some work from house current and some from batteries, etc., etc.

Marijuana joints and marijuana pipes burn pot. Under ideal circumstances, a vaporizer heats the pot but does not burn the pot. However, circumstances are not always ideal; vaporizers are well known to singe or burn the pot and that creates smoke that's not good for lungs. Factors underlying singeing and burning are comprised of: how dry the pot is; what “strain” it is; how it is prepared for vaping; how it is packed; the temperature setting of the vaporizer; even the altitude at which users vaporize their pot may affect burning, because air gets thinner the higher one gets. Air with its oxygen is required to singe or burn pot, but air is not necessary to inhale vaporized pot—other gases that do not support combustion would work just fine to inhale chemical vapor produced by sufficiently heated pot.

Disclosed also are novel vaporizers that derive heat from light, such as sunlight, which is concentrated via a curved reflector to an oven crafted to hold the pot, comprising apparatus to inhale the vapor that arises from the heated pot.

### Nosie™, French Inhaling Vaporizing, e.g., w/a Pax2

There is more than one way to enjoy vaporizing herb. For example, to eliminate all possible burning, inert gas can be used instead of air with its 20% or so oxygen to transport the herb's vapor into the user's lungs via the user's mouth, or via the user's nose, either with air's oxygen or without.

A “French Inhale” on a cigarette is or may be described thusly, similar to the way it is described on the Internet: a trick done with inhaled smoke (presumably of a cigarette, or joint). It involves sucking smoke from the cig into the mouth without inhaling it completely, then opening the mouth and pushing out the smoke with the tongue while inhaling it through the nose.

French inhaling looks cool, the herb “tastes” and feels different, and it prevents herbal-vapor from fouling the user's mouth parts comprised of teeth, tongue & cheek insides. Herb users will want to try “nosing it,” and many will want to buy Nosie™ as an alternate intake means for herbal vapor.

I invented Nosie in a proverbial “flash of genius” on Friday the 13th, 2017, my lucky numbered-day. Here's how my flash of invention occurred: I had just finished a fresh bowl of marijuana in my fantastic new Rose Gold Pax3, and I leaned back against my puffed up pillows, and I dosed-off for a catnap as I often do after lunch time which I like to skip for Pax-time—career inventors may be like that, you know, eccentric, tending to step to their own tune down the paths less taken. I awoke with quite a start and blurted out “French Inhale” so loud my two cats woke up too. I love it when I am awoken by a classic flash of genius, please pardon my immodesty, but that flash of genius is what inventors like me live for in significant part. I also have spent years in back-breaking research to finally be rewarded by discovering an invention worth patenting.

After being awakened with my “flash of genius,” I used my Pax multi-tool and a Q-tip to empty/clean my bowl and load up again. I don't usually load up again but I wanted to try cupping my nose to try a French Inhale from my Pax3.

“Vaping” seems to allow my unconscious mind more freedom to create. I've had multiple inventions via “flash of genius” both while sleeping and while awake; an image and or a word/phrase pops into my mind out of nowhere. That being said, there was a period of over ten years when I gave up tobacco and pot completely. (Giving up tobacco was pure hell!, pot, not-so-much, pot was no problem at all to give up compared to giving up tobacco.) During that 10+ year period without pot my creativity did not seem diminished, my creativity seemed about the same. With the advent of vaporizers, I was able to manage using pot once again. But I digress.

French Inhaling a vaporizer was everything I dreamed it would be, only better, because tasting is very much the nose's job—the nose adds “depth” & “flavor” to vaping. Any material, preferably somewhat pliable material, may be used for making Nosie that can be shaped or in any way made into the desired shape (a) to accommodate and seal or semi-seal in an opening for a user's nose (encompassing at least the user's nostrils), and (b) a cutout opening to effectively cover the output end of a vaporizer so as to allow the user to enjoy herb vapor through the nose, the air passing into the vaporizer via the input end which has the heating oven, to the output end for the user to inhale the vapor that is produced through his nose.

I've been experimenting cupping my hand, for example, and I like the way it feels more than by mouth, but it would be much easier & better with an anatomically designed nose piece (or nose cup) configured to fit a nose and adapted to cover the output of a user's preferred vaporizer.

Users could be encouraged to try cupping their hands or otherwise to experiment with and to see how much they enjoy the vape “nosing” experience. Once they try it, they will likely want to buy a Nosie to facilitate the nose vaping experience, an improved way to enjoy inhaling herb vapor. Also, a nosie promises to be more efficient and waste less vapor than making do with hand cupping.

As may be apparent to those skilled in the art, the embodiments described herein may be adapted to various kinds of vaporizers within the scope of the embodiments herein and possible claims therefore.

### Objects & Summary of Embodiments

A safer and healthier way to enjoy herbs: Vaporizer modes have been invented, disclosed herein, which allow a non-burning gas, such as carbon dioxide or nitrogen from commercially available cartridges, to pass over the heated pot instead of air to avoid the risk of the pot being singed or burned making smoke amidst the vapor. Health-wise, the use of non-burning gas instead of air is the safer way to vaporize weed; and you can crank up the temperature so as to get as much Vape as possible from the weed economically & efficiently and, of course, safely—health comes first.

One object of the present invention, embodiments of which may be called a “Thingy™”, e.g., Thingy, Thingy1™, Thingy2™or Thingys™, is to allow the use of a vaporizer while avoiding risk of smoke from burning or singeing the pot. Perhaps another more fitting name will emerge, but Thingy works for present patent disclosure as nothing preferable comes to mind. In case Thingy works out long-term for marketing purposes, Thingy etc. is hereby/will be claimed as trademarks. When this application is published, it will indicate the intention to use Thingy trademarks in commerce. I understand that the first step in acquiring a trademark is to use it as early as possible with a ™ so others are aware of one's trademark intentions.

In one embodiment, for a vaporizer having a heater, to heat herb such as pot, to vaporize one or more chemical components of the herb to allow a user to inhale the chemical vapor, what is disclosed is an anti-singe/anti-burn improvement mode to prevent singeing or burning of the herb as may happen if air is inhaled over the herb. One embodiment, Thingy1, may be integrated into a vaporizer so as to be one with the vaporizer. Another embodiment, Thingy2, may be a separate accessory to a vaporizer. Thingys are comprised of a non-burning gas supply, e.g., a carbon dioxide or nitrogen cartridge (such gases, certainly in the amount required to vape even a lot of pot, may have negligible or no adverse effect on health), and a regulator to release the gas via a gas control. The apparatus for Thingys may be contained in a box, such as a container to hold something, which may be made of plastic, metal, wood, glass, etcetera.

In embodiments described below, operating in an improvement mode, air is prevented from passing over the herb; instead of air, a non-burning gas passes over the herb to be inhaled so that the user does not risk inhaling any singed or burnt herb smoke along with the herb's chemical vapor.

“Mode” is a noun and means a manner and/or way and/or method of doing something and/or experiencing something, a manner of functioning or operating, a particular functioning arrangement or condition. Some embodiments of the present invention provide improvement modes for the use of herb in a vaporizer as described herein or made obvious from changes, modifications, etc. to the teachings hereof. It is the intent of the inventor to cover by the claims hereto all such modes and things stemming from these teachings.

It is an object of the present invention to disclose a vaporizer device having a heater, to heat herb such as pot, to vaporize one or more chemical components of the herb to allow a user to inhale the chemical vapor, an anti-singe/burn improvement mode to prevent singeing or burning of the herb as may happen if air is inhaled over the herb, comprised of: a non-burning gas supply, a lid, a regulator to release the gas, with a gas control, to be inhaled with the vapor via the lid, the improvement mode adapted to prevent air from passing over the herb and allowing instead the non-burning gas to be passed over the herb and inhaled, so that the user does not risk inhaling any singed or burned herb smoke.

It is an object of the present invention to disclose a vaporizer device accessory for a vaporizer device having a heater, to heat herb such as pot, to vaporize one or more chemical components of the herb to allow a user to inhale the chemical vapor, an anti-singe/burn improvement mode afforded by the accessory to prevent singeing or burning of the herb as may happen if air is inhaled over the herb, comprised of: a non-burning gas supply, a lid, and a regulator to release the gas, with a gas control, to be inhaled with the vapor via the lid, the improvement mode adapted to prevent air from passing over the herb and allowing instead the non-burning gas to be passed over the herb and inhaled, so that the user does not risk inhaling any singed or burned herb smoke.

It is an object of the present invention to disclose for use with a herb vaporizer having a heater to heat an herb to vaporize a chemical component of the herb to allow a vaporizer user to inhale the chemical vapor, an apparatus to facilitate inhaling through the nose comprising: a nosie apparatus having an opening to accommodate a user's nose and an opening, i.e., a cutout to cover the output of a vaporizer configured so that a user may cover a vaporizer device output with the nosie apparatus and when a user's nose is in the opening, all else being equal, the user may inhale vapor through the nose;

all else being equal meaning that the vaporizer is loaded and heated and ready to be used for vaping.

It is an object of the present invention to disclose for use with an herb vaporizer having a heater to heat an herb to vaporize a chemical component of the herb to allow a vaporizer user to inhale the chemical vapor, an apparatus to facilitate inhaling vapor through the nose comprising: a nosie apparatus having an opening to accommodate a user's nose and a cutout to cover the output of a vaporizer configured so that a user may cover a vaporizer device output with the nosie apparatus and when a user's nose is in the opening, all else being equal, the user may inhale vapor through the nose.

It is an object of the present invention to disclose for use with a herb vaporizer apparatus comprising magnets to help hold Thingy in place and to help hold Nosie in place.

It is an object of the present invention to disclose that, often when vaporizing herbs like marijuana, the pot gets singed or burned creating smoke; inhaling smoke is unhealthy. An improved vaporizer mode allowing a non-burning gas, such as carbon dioxide or nitrogen from small readily-available cartridges, to pass over the heated pot to inhale the herb's vapor instead of air to avoid the risk of the herb being singed or burned which makes smoke amidst the chemical vapor. Without air's oxygen, burning and smoke are just not possible no matter how dry the pot is or how hot the pot gets.

It is an object of the present invention to furthermore disclose that, finally, apparatus to facilitate taking a French inhale from a herb vaporizer has been invented! French inhaling is cool, the tobacco or other herb “tastes” and feels a lot enjoyable (most of “flavor” is in the nose) and French inhaling prevents herbal-vapor from “fouling” the user's mouth.

It is yet another object of the present invention to disclose one embodiment wherein vapor carried by a non-burning gas, which is delivered using Thingy, may be French inhaled using Nosie™. Two complementary embodiments, which may be used at the same time while vaporizing, of the present vaporizer invention are disclosed.

### BRIEF DESCRIPTION OF THE DRAWINGS

All drawings Figs. may have drawing parts not drawn to scale of other parts. (For illustrating purposes, the PAX LED user interface may be shown toward one end or the other of the PAX2.)

FIG. 1A illustrates a nosie 501 to facilitate French Inhaling (Inhaling with or without an initial capital letter mean the same; same with Nosie and nosie) from a vaporizer. Nosie 501 apparatus can be configured and or adapted to work with many different vaporizers.

FIG. 1B illustrates a side view nose and Pax2 513.

FIG. 1C illustrates a front view nose and Pax2 525.

FIG. 1D illustrates a front view nose, nosie 501 and Pax2 525.

FIG. 1E illustrates a side view nose, nosie 501 and Pax2 513.

FIG. 1F illustrates a nosie 501 with magnets 511 and with a nasal cannula 503.

In FIGS. 1 to 5A, like parts are numbered once (except, like parts are numbered in both FIGS. 2 and 5A). Like parts may also be numbered alike elsewhere.

FIG. 1 depicts a Pax2 1, a hugely successful embodiment of a contemporary vaporizer, a modern miracle of ingenuity.

FIG. 2 depicts Thingy2 31, an accessory for a vaporizer, an embodiment of the present invention adapted to a Pax2 1.

FIG. 2A depicts another embodiment similar to FIG. 2 but it doesn't have a gas box 43.

FIG. 3 depicts Thingy2 31 mounted on a Pax2 1 ready to “toke-up” once herb is added & heated; parts as above. In common parlance & practice in the world of users, when one tokes-up one takes a few hits.

FIG. 4 depicts another embodiment of the Thingy invention called Thingy1™.

FIG. 5 depicts a light powered vaporizer embodiment.

FIG. 5A depicts a light powered vaporizer embodiment with Thingy

FIG. 6 is FIG. 2 with the addition of a safety valve 151 illustrated by way of example as shown in lid 33 but it could handily be fitted elsewhere. Magnets 171 are illustrated.

FIG. 6A is an oblique close up view of a part of safety valve 151, opening 153, with a part of FIG. 6's lid 33, lid 33's part 161.

FIG. 7 consists of Thingy2 31 from FIG. 2, flipped vertically and horizontally, and FIG. 1A's nosie 501 installed on a Pax2 525.

FIG. 7A has been compiled to serve as the selected figure for page one of the patent, and nothing more; FIG. 7A adds no new matter.

### DETAILED DESCRIPTION

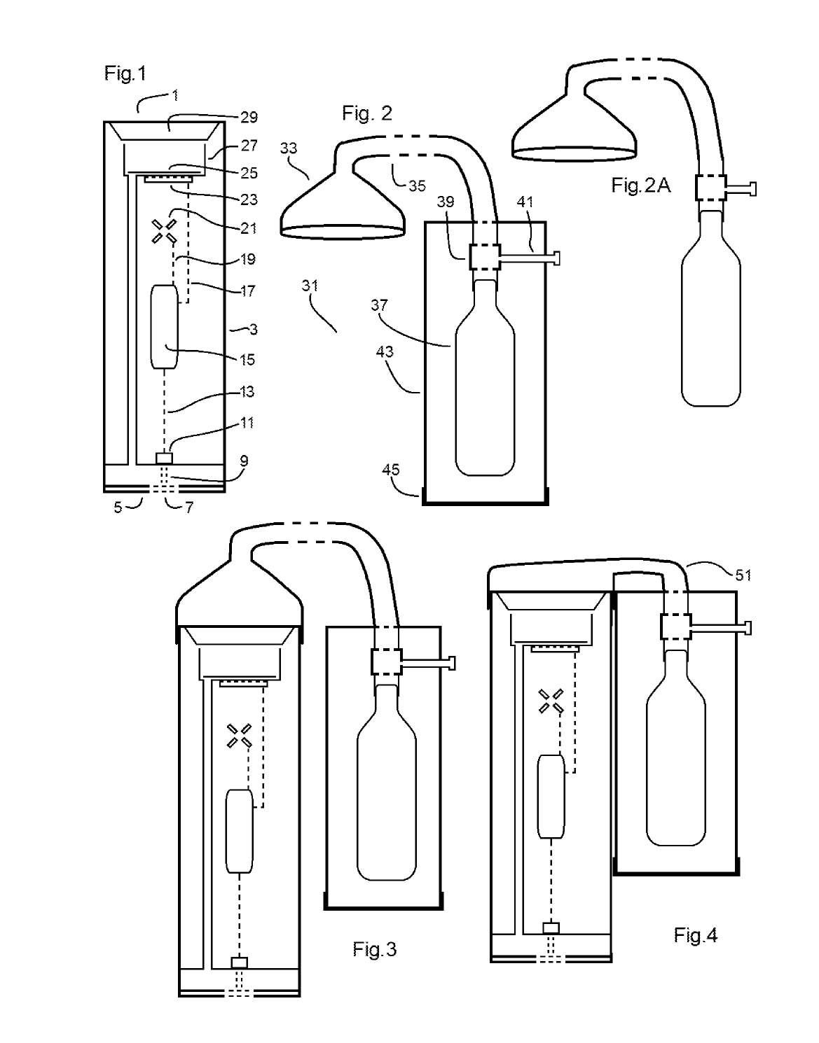

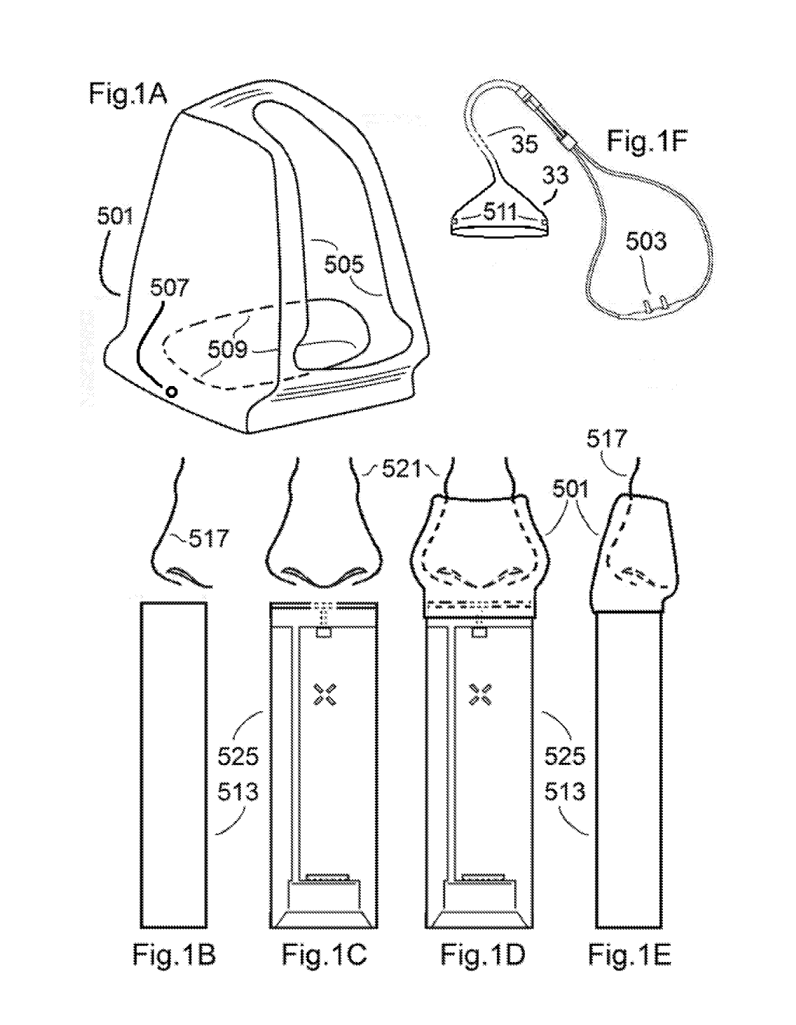

In FIG. 1, the itemized parts are comprised of:
1, a Pax2 1;
3, body case 3;
5, mouth piece 5;
7, draw opening 7,
9, mouth/controller switch linkage 9;
11, controller switch 11;
13, switch connection 13;
15, Pax2's persona/soul 15 (meaning the ingenious electronic “heart” of Pax2);
17, heater connection 17;
19, LED interface connection 19;
21, LED interface 21;
23, heater 23;
25, oven plate 25;
27, oven, and
29, cover 29.

FIG. 1 depicts a Pax2, a hugely successful embodiment of a contemporary vaporizer, a modern miracle of ingenuity. Different embodiments of the improvement modes described herein may be called “Thingy.” Such accessory Thingys may be configured for use with a Pax2 or adapted to fit any other vaporizers that heats the marijuana (or tobacco, other herb, etc.). The Pax2 is utilized in the drawings and disclosure by way of example and is not meant to be limiting in any way.

The Pax2 (and now Pax3), is the best delivery system for marijuana ever invented, and it is so beautifully presented & cleverly engineered, simply elegant in the purest Einsteinian way. After I showed my new gold Christmas gift Pax2 to my neighbor, she was so impressed that she bought one in platinum around the corner in the tiny smoke shop the next day. Now he says he'll never roll another joint. Now that's a hugely successful product—pure genius of a product! I am proud to stand on Pax2's shoulders and contribute to safer and happier vaporizing for a growing number of modern users using vaporizers—especially users like me with lungs ravaged by decades of tobacco and pot smoking.

Referring now to FIG. 1A, a cozy nosie 501 (aka, CozyNosie™) is a nose piece, a nose cup, constructed somewhat in part, for example, in at least one embodiment, of non-rigid material, so that the non-rigid part of nosie 501 that covers or “cups” a vaporizer user's nose, such as a Pax2 user, will “form-fit” and mostly or completely cause the user's nose to not allow ambient air to be sucked in through an unsealed part of nosie 501 against the user's face while engaging nosie 501 to inhale vapor via the user's nostrils.

In one embodiment, nosie 501 is configured to allow a user to control how much air may be sucked in while inhaling vapor through the nose by allowing air leakage. Cutout 509 is illustrated in part as an opening with dashed lines and solid lines as may be seen through user nose opening 505. The shape of Nosie 501 in FIG. 1A is illustrated by way of example; a nosie can be in the shape of various designs and myriad shapes. One thought toward efficiency is to make it a comfortable but tight fit to lessen air space when nosie 501 is being utilized.

Instructions for use of nosie 501 may be comprised of: Fill Pax2 bowl with herb of your choice and press mouth piece to turn Pax2 on; mount nosie 501's vaporizer cutout 509 on top of the mouth sucking end of the Pax2 and urge cutout 509, or “snuggle” it, into position to cover the mouth end (the removable mouth piece may be left in place or removed if preferred); with provision made in a vaporizer to which nosie 501 is to be (temporarilary) attached (or affixed, or “snapped” into place), magnetic catches, only one is shown in FIG. 1A, magnet 507, may be used to help hold nosie 501 (temporarily) in place; when all is ready, insert your nose in the accommodating nose opening 505 of nosie 501, press nosie 501 to make an air-tight seal as you may like, and inhale cautiously and gently through your nostrils to begin to learn how you like using nosie 501 best, most comfortably for your nose & your enjoyment; then, remove nose from CozyNosie, lean back and enjoy, life is so beautiful & precious and it expires all too soon. Suggestion: at this point, Count your Blessings and offer up Thanksgivings.

Referring now to FIGS. 1B, 1C, 1D and 1E—what is illustrated is a front view Pax2 525 in FIGS. 1C & 1D and a side view Pax2 513 in FIGS. 1B & 1E. An outline side view nose 517 is illustrated in FIGS. 1B & 1E. A front view nose 521 is illustrated in FIGS. 1C & 1D. In FIG. 1D nosie 501 is illustrated in place, with cutout 509 from FIG. 1A covering the mouth end of Pax2 525, and with part of front view nose 521 which is illustrated in part with dashed lines behind nosie 501.

In FIG. 1E nosie 501 is illustrated in place on side view Pax2 513 with cutout 509 from FIG. 1A covering the mouth end of Pax2 513, and with part of side view nose 517 which is illustrated in part with dashed lines behind nosie 501.

In Fig. IF another embodiment is illustrated with a lid 33 and gas connection 35, FIG. 2, as shown connecting lid 33 to a medical-type oxygen nasal cannula 503. With the lid 33 covering the mouth end of a vaporizer, the user can, for example, inhale through the nose via an oxygen-like cannula for hands-free nose inhaling. Using a nasal cannula 503 may discretely conceal vaping in non-vaping locals, but, to avoid the tell-tale odor give-away, the user should not let any tasty vapor be wasted. A magnetic catch, magnets 511 illustrated in FIG. 1F, may be used to help hold lid 33 in place.

As is shown & discussed in association with FIG. 1, some of the inner working of a Pax2 is shown in FIGS. 1C & 1D. LED interface 21 of FIG. 1 is illustrated in FIGS. 1C & 1D more correctly positioned near the mouth end of Pax2 525.

Pen style and other styles and shapes of vaporizers for tobacco and marijuana, for example, for either herb plant or oil vaping, may be configured with a mouth piece that works well for the nose as well. Or a nosie apparatus as described above could be configured for pen style or other style vaporizer. Yes, nosing it works well with either plant material or oils therefrom or synthetically made.

FIG. 2: the itemized parts are comprised of:
31, Thingy2 31, an embodiment of the present invention adapted to a Pax2 1;
33, lid 33, an essentially air tight lid to slip on and cover the oven-end of a Pax2, the air intake end which would allow air in if the lid 33 were not in place.
35, gas connection 35, connects lid 33 to gas box 43;
37, non-burning gas cartridge 37;
39, regulator 39;
41, gas release 41;
43, gas box 43; and,
45, gas box foot 45, for standing up Thingy2 31.

Lid 33, depicted in FIGS. 2 and 3, may be interchangeable with lid assembly 51 depicted in FIG. 4, meaning lid 33 or lid assembly 51 can be used similarly on a Pax2 to cover the air-intake end. The lid 33 may be configured and shaped to fit other vaporizers, for example, a conically shaped vaporizer, such as one that may be shaped similar to a pen.

Because air with its oxygen is required for burning (combustion), to avoid the risk of any burning, Thingys provide a non-burning gas instead of air to pass over herb while heated to produce vapor which is to be inhaled by the user. Air is not needed to heat & vaporize the herb. Conveniently sized commercially available gas cartridges are discussed herein by way of example, but that is not meant to be limiting—gas could come otherwise for purposes of safer vaporing as disclosed herein.

In hospital or hospice facilities for health conditions that respond well to marijuana, for example, a gas such as nitrogen could be made available bedside throughout the facility like oxygen, and made available alongside the individual O₂ supply ports that patients may require. In this embodiment, a patient may vaporize pot and other herbs without fear of burning which could be particularly hazardous because oxygen may be in use nearby.

The Pax2 vaporizer discussed herein by way of example is used per manufacturer's instructions and suggestions with exception. An exception is that air is prevented from passing over the pot in the heated oven using, for example, lid 33 in FIGS. 2, 2A & 3, or lid assembly 51 in FIG. 4.

Referring now to FIGS. 2, 2A, 3 and 4: A supply of non-burning gas is provided, instead of air, via user controlled gas release 41 via a regulator 39 via gas cartridge 37 to supply its non-burning gas. While a user can still inhale, s/he can also sit back and let the pressurized gas from the cartridge enter her/his lungs with or without inhaling, or with less inhaling.

Right after turning on the Pax before it heats up much, one can purge the air out of the insides of the Pax by releasing an amount of gas to displace or push the air out to avoid burning or singeing. This is important especially if the user puts down the Pax and gets a little distracted before the user starts vaporizing, allowing time to burn; but if the air has been purged, no problem.

FIG. 2A depicts a possible embodiment by way of example; FIG. 2A is the same as FIG. 2 but without gas box 43. A box (e.g, a container to hold the cartridge) is not used to hold the gas cartridge 37, gas release 41 and regulator 39 which is connected via gas connector 35 to lid 33.

FIG. 3 illustrates FIG. 2's Thingy2 31 in place mounted on a Pax2 1 from FIG. 1.

FIG. 4 depicts another embodiment called, Thingy1, which is also adapted to a Pax2 1; the itemized parts in FIG. 4 are comprised of snuggly lid assembly 51; other parts as above. As may be seen as missing is a gas connector 35 which, in FIG. 4, is part of lid assembly 51. FIG. 4 depicts an embodiment of the Thingy invention broadly called Thingy1. Thingy1 may be integrated, non-removable (fixed) or removable, with a vaporizer so as to be one with the vaporizer; i.e., FIG. 4's Thingy1 may be detachable. Another embodiment, Thingy2, may be a separate accessory to a vaporizer.

The embodiment depicted in FIG. 4 may be available as a single handheld unit that may be handily separated into two handheld pieces, the Pax2 vaporizer piece and Thingy1.

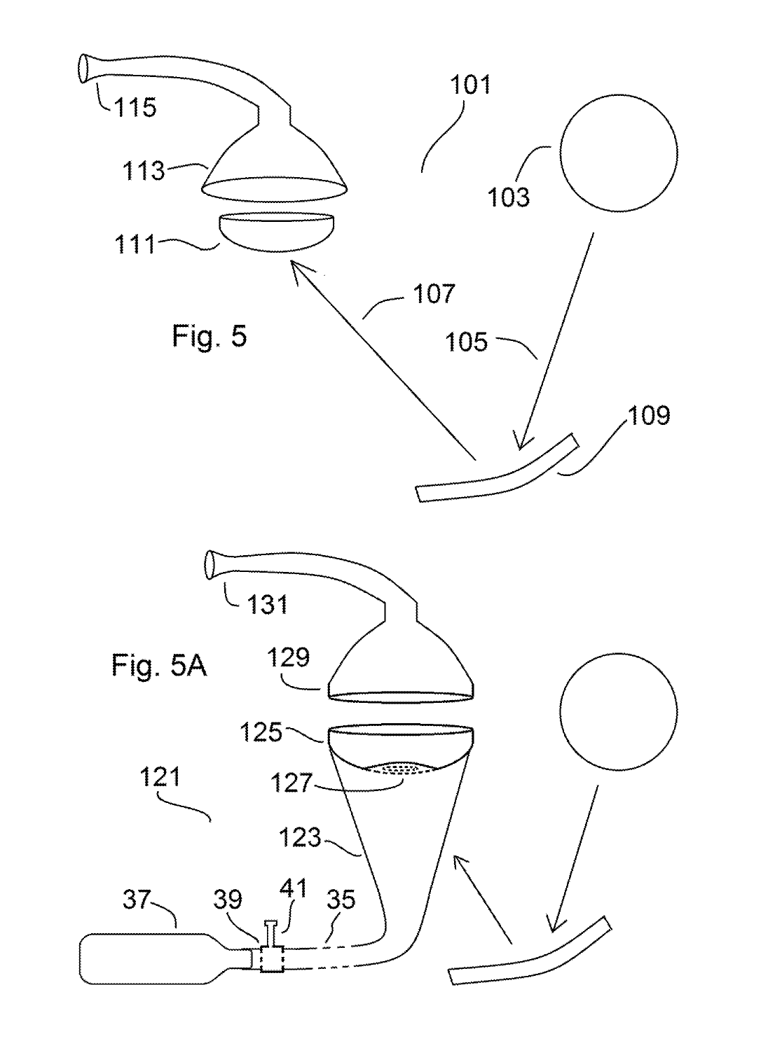

FIG. 5 depicts a light powered vaporizer comprised of the following parts:
101, sunlight powered vaporizer 101;
103, the sun;
105, sunlight from the sun 105;
107, reflected sunlight 107;
109, reflector 109;
111, pot heater 111;
113, sun lid assembly 113;
115, sun lid mouth piece 115.

FIG. 5A depicts a light powered vaporizer with a non-burning gas supply comprised of the following parts not found in FIG. 5:
35, gas connection 35, connects lid 33 to gas box 43;
37, non-burning gas cartridge 37;
39, regulator 39;
41, gas release 41;
121, light powered vaporizer with non-burning gas supply 121;
123, a conical-like shaped transparent pot oven holder 123;
125, sun pot oven 125;
127, pot oven part for ventilating 127;
129, sun-lid 129, an essentially air tight lid to slip on and cover the sun pot oven;
131, sun lid mouth inhale port 131.

FIG. 5 depicts a light powered vaporizer 101 embodiment which requires no electricity. Sunlight 105 from sun 103 shines on curved concave reflector 109 and reflected sunlight 107 is concentrated to shine on the pot heater 111 to heat the pot (not shown) so a user (not shown) can inhale via sun lid mouth piece 115 vapor rising into sun lid assembly 113 from the pot (not shown) being vaporized in the pot heater 111. Sun lid assembly 113 and pot heater 111 may be joined one above the other via, for example, a swing away hinge (not shown).

FIG. 5A depicts a light powered vaporizer embodiment with a non-burning gas supply 121. Gas connector 35, gas cartridge 37, regulator 39 and gas release 41 are configured like, and operate in accord with, the disclosure for FIG. 2 above.

For embodiments associated with FIGS. 5 & 5A, a user may hold things including the curved reflector 109, sun lid assembly 113 and light powered vaporizer with a non-burning gas supply 121 to enjoy herb, or these things may be configured to be set down or enclosed in a suitable holder to make vaporizing convenient, user-friendly (this “configured” notion is similar for other embodiments as well).

In FIG. 5A, a conical-like shaped transparent pot oven holder 123 is constructed of transparent material so that concentrated light can reach sun pot over 125 to heat pot (not shown) in the oven 125. Pot oven part for ventilating 127 allows non-burning gas to flow through openings (illustrated with the little dashes of ventilating 127) so the user can inhale non-burning gas with pot vapor that has been vaporized by the heat from sunlight from curved reflector 109. Sun-lid 129 is essentially a relatively air tight lid to slip on and cover the sun pot oven 125. Sun lid mouth inhale port 131 is for inhaling the smoke-free vapor.

The use of non-burning gas avoids the health risk of inhaling smoke when vaporizing no matter how dry the pot is or how hot the pot gets. What has been disclosed are safer and healthier embodiments to enjoy herbs.

### Safety Valve for Thingy

There is concern that the regulator 39 in FIG. 2 and elsewhere that releases a gas via a gas control 41 releases so much gas that the pressure builds up beyond a point that may damage a user's lungs. In this embodiment, a safety pressure sensitive release valve 151 may be installed in lid 33. Alternatively, valve 151 may be located in gas connector 35.

Opening 153 allows excess gas to escape if the gas pressure increases past a set (or settable) safety limit. The escape is allowed when the gas pressure increases sufficiently to push open gas flapper 155 which pivots at hinge point 157 to open and close depending on the level of the gas pressure.

Especially given what vaporizers may be used for—to get stoned—there is such concern because users may get too stoned and think they may have an even more stoned experience if they held their mouth over the vaporizer's mouth piece to seal it off and then release enough gas to expand their lungs artificially with herbal vapor. It can be dangerous to over-expand one's lungs, and therefore use of a safety valve, such as valve 151 as shown and described, is another embodiment.

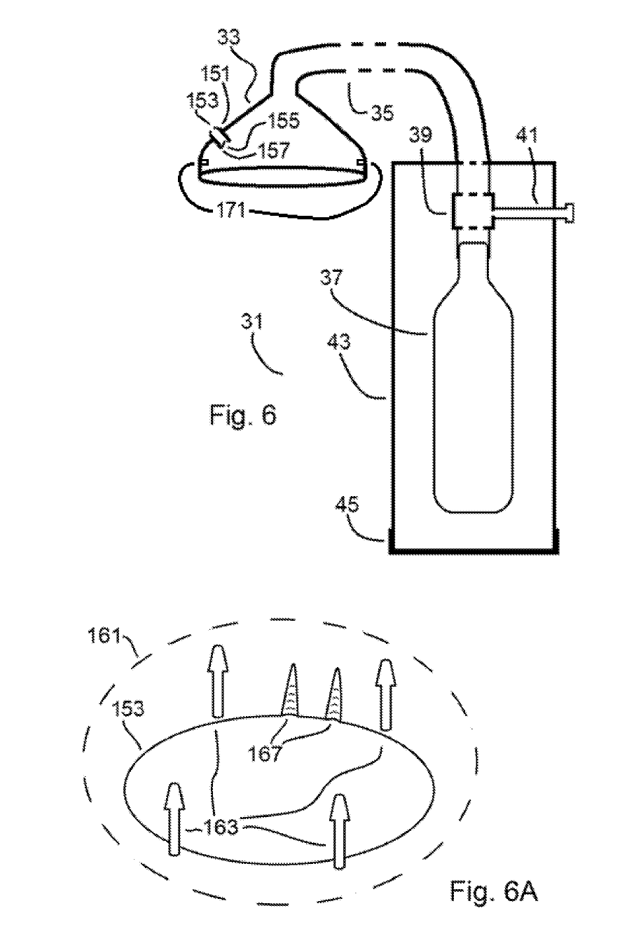

FIG. 6 is FIG. 2 with the addition of a safety valve 151 illustrated by way of example as shown in lid 33 but valve 151 could handily be fitted elsewhere. In FIG. 6's embodiment, by way of example, a magnetic catch, two magnets 171 are illustrated. With provision made in a vaporizer to which Thingy2 31 is to be (temporarily) attached (or affixed) as illustrated in FIG. 3, magnets 171 may be employed to hold Thingy2 31 in place during vaporizer operation.

FIG. 6A shows an outline 161 around the portion of the surface of lid 33 containing opening 153. To prevent a user from blocking, with his finger for example, the opening 153 to defeat the safety valve to try to get more stoned, the four posts 163 shown, and the two tapered-furrows 167 shown, tend to prevent such blockage. More or fewer posts 163 and tapered furrows 167 may be utilized.

For medical purposes, pressurizing lungs to expand them beyond what a person can expand unassisted is called, Intermittent positive pressure breathing (IPPB) therapy. It is known that IPPB may be useful in patients like me with COPD. However, because of machine costs and high administering costs due to extreme FDA “caution” reasons requiring a licensed therapist to continuously monitor the patient, IPPB fell out of favor. Before I knew any of that, I discovered on my own that I could safely administer this helpful therapy all by myself with a mouth piece used to mist medicine with a Nebulizer, a balloon and an oxygen tank. I set the regulator on the oxygen tank to 6 liters, similar to the output of a Nebulizer, attach a toy party balloon as a safety regulator (like a “governor”) on one side of the mouth piece, load the mouth piece with, e.g., albuterol solution, seal my mouth on the other side of the mouth piece and expand away when I open the tank's valve. If the balloon starts to blow-up, that signals enough expanding.

In one embodiment herein, a safety pressure sensitive release valve 151 may be selected to allow a safe level of lung expanding.

Having advanced COPD, my observation is that it is a mental matter as well as it is about damaged lungs. Breathing is autonomic, part of the nervous system responsible for control of essential the bodily functions not consciously directed, along with heartbeat, and digestive processes. If a patient has had breathing panic attacks, which are like waterboarding, he will thereafter fear panic attacks and high anxiety results—marijuana to the rescue.

According to intelligent doctors, marijuana is far safer and more effective than hundreds of prescription drugs for a large variety of ailments, like anxiety, depression, personality disorders, PTSD, COPD, pain, restless leg syndrome, sociopathic behavior, even war and other aggressive behavior, etc., and woes of almost any sort. Many believe that marijuana is a miracle drug, a blessing if you will, especially in the new strong strains that are now available.

I'll end this spec with this story, how I invented Thingy by not “exploding” my head, my thinking process that gave birth to my Thingy embodiment. While using oxygen from an E tank for IPPB therapy as I often do to aid my breathing, I was also vaping with my PAX2. I looked at my oxygen tank thinking, maybe I could inhale my vapor with oxygen instead of air, kinda like two birds with one stone, I could get a little boost of O₂ while absorbing the herb's vapor. But in a flash at that moment, I knee-jerk slapped my forehead thinking and visualizing—stupid, you could blow your head off passing straight O₂ out of the tank over heated, possibly burning or smoldering, herb!

This “flash” occurred to me because I had observed many times in the past that my pot got singed or burned and it had a black burnt color and or odor after it was vaporized. Thus, using pure oxygen could be “explosive” and dangerous. Perhaps I could get TV's “MYTHBUSTERS” to try an experiment—they seem to like exploding experiments.

Be that as it may, I soon realized that, while taking oxygen using a nasal cannula, I had safely simultaneously vaped many times as the O₂ doesn't pass over the hot pot if it is being delivered by nasal cannula. Somewhere along here I invented using a nasal cannula to deliver vapor through the nose. My observation is that the vaping experience is not noticeably different with an O₂ boost whether I take the vapor by mouth or by my nose.

Another observation: I prefer vaping by French inhaling but it would be greatly facilitated and less wasteful of good vapor if I had a nosie 501. For example, I could handily French inhale using only one hand.

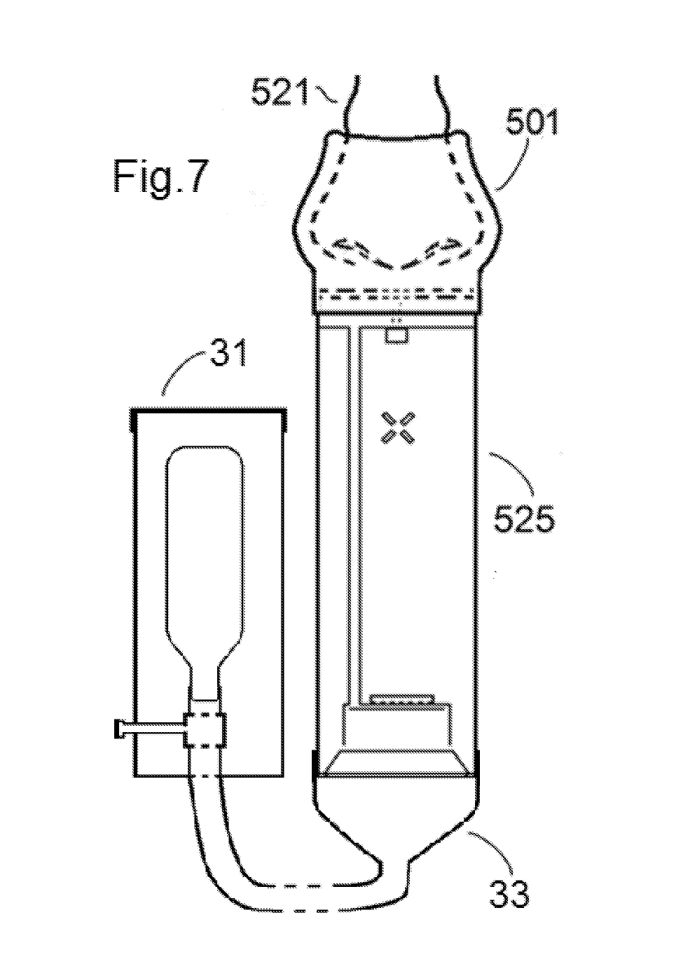

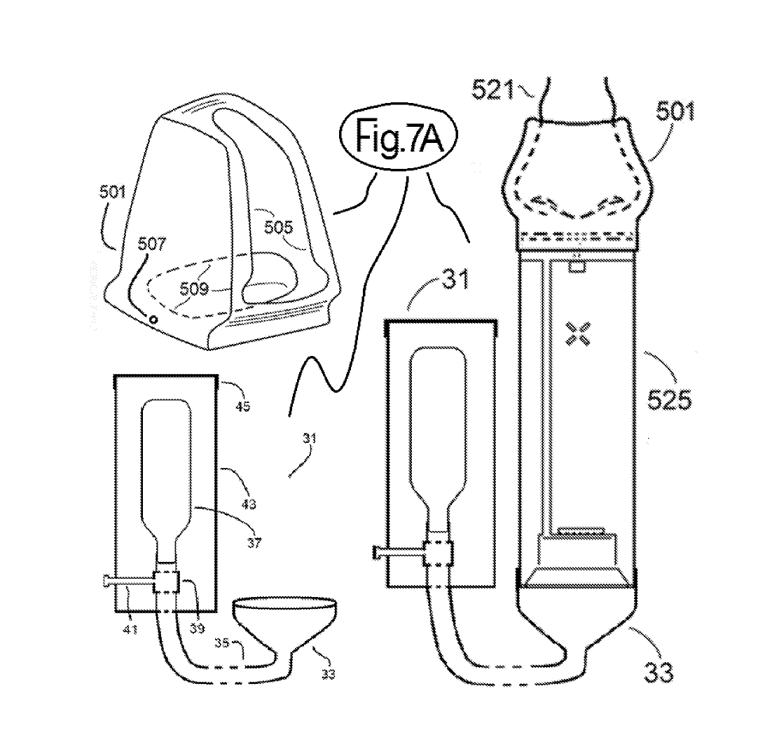

FIG. 7 illustrates simultaneously use of a nosie 501 as described above and a Thingy2 31 from FIG. 2 as described above on a Pax2 525. In this embodiment, everyone may handily enjoy one-handed French inhaling safer smoke-free vapor from their vaporizers, the best of all worlds in my subjective view.

FIG. 7A has been compiled to serve as the selected figure for page one of the patent, and nothing more; i.e., FIG. 7A is purely for illustration on page 1. FIG. 7A consists of Thingy2 31 from FIG. 2, flipped vertically and horizontally and FIG. 1A's nosie 501, both installed on a Pax2 525 as shown in FIG. 7. Each of FIGS. 1A, 2 and 7 are described separately.

I claim:

1. An accessory for a vaporizer which heats an herb to vaporize at least one chemical in the herb to allow a user to inhale the herb's chemical vapor, an anti-singe/burn accessory, to prevent smoke from singeing or burning herb from air passing over the heated herb and being inhaled with the vapor, comprised of:
a non-burning gas supply,
a lid,
said lid configured with a regulator to release with a gas control the gas to be inhaled with the vapor via the lid,
the accessory configured to prevent air from passing over the heated herb and allowing instead the non-burning gas to pass over the herb and be inhaled so that smoke is prevented.

2. For a vaporizer which heats an herb to vaporize at least one chemical in the herb to allow a user to inhale the herb's chemical vapor, an anti-singe/burn improvement mode, to prevent smoke from singeing or burning herb from air passing over the heated herb and being inhaled with the vapor, comprised of:
a non-burning gas supply,
a lid,
said lid configured with a regulator to release with a gas control the gas to be inhaled with the vapor via the lid,
the mode adapted to prevent air from passing over the heated herb and allowing instead the non-burning gas to pass over the herb and be inhaled so that smoke is prevented.

3. For a vaporizer comprising a heater to heat herb to vaporize at least one chemical in the herb and configured so that a user can inhale the herb's chemical vapor after the herb is heated, an accessory to facilitate inhaling vapor through the nose comprised of:
apparatus comprising an opening to accommodate a user's nose and
an opening adapted to cover the output of a vaporizer,
said apparatus adapted to allow a user to insert her or his nose in the apparatus and to cover with said apparatus the output of a vaporizer ready to be used to facilitate inhaling through her or his nose.
